(19) United States
(12) Patent Application Publication
(10) Pub. No.: US 2007/0239127 A1
(43) Pub. Date: Oct. 11, 2007

(54) DISPOSAL WRAPPER FOR USED FEMININE PRODUCTS

(76) Inventor: YVONNE BRIDGES, Bear, DE (US)

Correspondence Address: CONNOLLY BOVE LODGE & HUTZ, LLP, P O BOX 2207, WILMINGTON, DE, 19899, US

(21) Appl. No.: 11/278,844

(22) Filed: Apr. 6, 2006

(51) Int. Cl. A61F 13/15

(52) U.S. Cl. 604/385.13; 604/385.02; 383/70

(57) ABSTRACT

A disposal wrapper for used feminine products, such as sanitary napkins and tampons, has a fluid-impervious wrapper in sheet form with a deformable wire at one edge. A used feminine article placed near the center of the wrapper is encircled by the wrapper and enclosed therein by twisting the wire Optionally, a dispensing container for multiple disposal wrappersmay be provided.

### BACKGROUND OF THE INVENTION

1. Field of the Invention

This invention relates to a sanitary disposal mechanism and method for used feminine articles, and specifically used tampons and sanitary napkins. A dispensing carton or carrier for the improved disposal mechanism is also provided.

2. Background Art

It is important to dispose of used sanitary napkins and tampons in a hygienic manner to prevent the leakage of menstrual fluid, as well as the permeation of undesirable odors onto fabrics and into the air. Many women dispose of their used feminine articles in a trash receptacle in a public area. The contents of such receptacles are often open to public view, and occasionally contain no bag or protective lining. Thus it is common for a woman to either wrap or conceal a used feminine article immediately prior to discarding in the trash receptacle, or retain a used article in her purse or pocket to dispose of outside of the ladies' room.

Because many women dispose of a used feminine article in public trash receptacles or carry the used articles out of public rest rooms with them, it is most desirable to dispose of a used feminine article in a manner which keeps the menstrual fluids within the used article, preventing leakage as well as preventing transmission onto hands, clothing, or other items, and containing the unpleasant odors of a used article.

Many publications have described feminine articles such as sanitary napkins and tampons having an individual wrapping or packaging made from a fluid impervious material. Commonly the material is a thermoplastic, such as polyethylene film or polypropylene film, and treated paper materials and laminate materials made of polyethylene film and paper have also been described.

Several disposal methods for used feminine articles have been suggested. One disposal method incorporates a disposal means into the feminine article itself. For example, U.S. Pat. No. 4,605,403 describes a disposal bag which is affixed to a sanitary napkin. This patent teaches that because the bag is integral to the sanitary napkin itself, the bag is easily accessible and may be conveniently removed to deposit a soiled article within it for disposal. Similarly, U.S. Patent Application Publication No. 2003/0073970 describes an individual package containing a feminine product in a bag enclosure which, among other features, can be separated into two parts so that one section serves as a disposal means.

Other disposal mechanisms have been described whereby the wrapper used to protect a clean feminine article may be later used to dispose of a soiled article. U.S. Pat. No. 4,735,316 describes a packaging for a fresh article which is pleated so that after the article is removed, the packaging can be expanded into a bag for a used article. Along the same lines, U.S. Patent Application Publication No. 2003/0116462 describes a pouch configuration for a clean article which may be resealed after opening, enabling a woman to place a used feminine article in the pouch and close it once the clean article is removed from the pouch.

None of these disposal means can be carried independently from the feminine article, nor can they be manufactured or sold independently of the feminine article. Because they are not independent of the feminine article itself, these disposal devices also can not be readily dispensed from a dispenser carton immediately prior to use.

Various independent disposal means for used sanitary products, which are in a bag, pouch, or container form, have also been described. U.S. Pat. No. 6,702,116 discloses a disposal container made by folding a liquid-resistant sheet. According to this invention, in order for a woman to use the disposal container, the woman must first assemble the disposal container by folding a liquid-resistant sheet in multiple folding steps, which may be labor and/or time-intensive. U.S. Pat. No. 6,402,727 also describes a disposal cover for a sanitary article which requires folding. The disposal cover of this invention is folded around a used article, and tape-fastening means are used to secure the folded flaps of the cover around the used article. Each of these disposal mechanisms requires more than two folding steps. In addition, folding the liquid-resistant materials in the manner described by each invention results in multiple seams from which menstrual fluid could potentially leak and from which unpleasant odors may escape the disposal packaging.

Several U.S. Patents describe creating an enclosure around an article by utilizing deformable wire means, as compared to folding or adhesive closure means. U.S. Pat. No. 4,267,768 discloses a reclosable thermoplastic bag with a pair of encapsulated thin aluminum wires on opposite ends of the bag. The wires are covered in thermoplastic and integral to the sidewalls of the bag. By twisting the wire means together at the open end of the bag, all of the thermoplastic material making up the open end of the bag is gathered together and the bag is closed.

U.S. Pat. No. 5,180,229 and U.S. Pat. No. 6,585,413, also disclose bags having a pair of plastic-covered wire closure means. The wires of these inventions are secured horizontally on the front and rear walls of the open end of the bag, extend horizontally beyond the length of the bag, and are covered with covering material to prevent the ends of the wires from puncturing the bag. The bags of these inventions are closed by bending the wires inwardly to close the open top of the bag, or by rolling the top of the bag and bending the extended wire means to form a clasp. These inventions are less economical of a containment and enclosure means, and more difficult to manufacture, than a closure means which contains only one thermoplastic sheet, and one wire means, which need not be covered either by thermoplastic or covering material.

### SUMMARY OF THE INVENTION

In a first aspect of the invention, a disposal wrapper for used feminine articles, such as tampons or sanitary napkins, is a fluid-impervious sheet having a front face, a bottom face, a length, a width and at least one edge, that has a deformable wire or twist tie at the at least one edge. The fluid-impervious sheet preferably is formed from a polymer or biodegradable polymer or a treated paper or fabric, and the wire preferably is formed from a thermoplastic coated metal, such as bendable iron. The deformable wire may be attached to the edge of the sheet with adhesive, or may be integrally formed in the edge, such as where the sheet has multiple layers forming a laminate and the wire is held between two such layers

The wrapper fully encloses the used feminine article, and may be easily discarded in a trash receptacle, or carried in a woman's purse to dispose later, without leaking menstrual fluids or unpleasant odors.

The sheet may define one or more fold lines so that the sheet may be folded in at least one fold configuration selected from the group consisting of: “C”-fold, “V”-fold and “Z”-fold. Such fold lines make it possible to store multiple sheets in a dispenser carton or container. Such fold lines also can assist a user to place a feminine article on one face of the sheet before wrapping or encasing such article in said sheet.

In a second aspect, a method of disposing a used feminine article includes the steps of (a) providing a disposal wrapper having a fluid-impervious sheet having a front face, a bottom face, a length, a width and at least one edge, and a deformable wire at the at least one edge, (b) placing the used feminine article on one face of the sheet, (c) encircling the used feminine article with the sheet, and (d) securing the sheet over the used feminine article by twisting the deformable wire. The sheet may be rolled over the used feminine article may be rolled to encircle the article. Each end of the deformable wire may be twisted in an opposite direction, such as the twist for a candy or taffy wrapper. Alternatively, each end of the deformable wire may be twisted to form a knot therein.

In a third aspect, a dispenser carton for disposal wrappers for used feminine products includes one or more walls defining a volume for holding multiple disposal wrappers, wherein at least one wall is a top wall defining an aperture to permit disposal wrappers to be pulled out of the volume. A plurality of disposal wrappers are held in stacked relation within said carton volume. A single wrapper may be pulled from the carton through the aperture. Preferably, the disposal wrappers are folded in a fold configuration selected from the group consisting of “V”-fold, “C”-fold and “Z”-fold.

Further objects and advantages of the invention will be readily apparent to those skilled in the art from the following detailed description, taken in conjunction with the annexed sheets of drawings on which are shown several preferred embodiments of the invention.

### BRIEF DESCRIPTION OF THE DRAWINGS

FIG. 1 is a dispenser carton for dispensing the feminine article disposal wrappers of the present invention;

FIG. 2 is a bottom plan view of the feminine article disposal wrapper of the present invention;

FIG. 3 is a top plan view of the feminine article disposal wrapper of the present invention, containing a used tampon to be enclosed and discarded;

FIG. 3A is a top plan view of the feminine article disposal wrapper of the present invention, containing a used sanitary napkin be enclosed and discarded;

FIG. 4 is a profile of the feminine article disposal wrapper folded or compressed in a “Z” formation prior to dispensing;

FIG. 5 is a profile of the feminine article disposal wrapper folded or compressed in a “V” formation prior to dispensing;

FIG. 6 is a profile of the feminine article disposal wrapper folded or compressed in a “C” formation prior to dispensing;

FIG. 7 is a side elevational view showing a used feminine article enclosed in the disposal wrapper of the present invention, secured by twisted wire means at each end whereby the twisted wire means forms a closure at each end; and

FIG. 8 is a side elevations view showing an alternate view of a used feminine article enclosed in the disposal wrapper of the present invention, secured by twisted wire means at each end whereby the wire is twisted in opposite directions at each end.

### DESCRIPTION OF THE PREFERRED EMBODIMENTS

The present invention is a feminine article wrapper for use in disposing of used feminine articles in a sanitary and convenient manner.

Referring first to FIGS. 2 and 3, wrapper 10 has a sheet having a top face 12 and a bottom face 14. Each sheet has a length and a width and has side edges around its periphery. Possible dimensions are a length of about 6 to 10 inches and a width of about 4 to 10 inches. The wrapper 10 preferably is formed from a fluid-impervious material, such as a thermoplastic film or sheet, a nonwoven layer coated with a thermoplastic, and a laminate sheet of a thermoplastic, a nonwoven, a woven with one or more adhesives or coatings.

Thermoplastic polymers include polyolefins, polyamides, polyesters, acrylic polymers, polyvinyl chloride, polyvinyl acetate and copolymers and blends thereof. More desirable are polyolefins, olefin copolymers and blends thereof. Polymers may include homopolymers, copolymers, terpolymers and modifications thereof Films containing a suitable polymer can be produced with conventional methods, such as casting and blowing processes.

The fluid-impervious material may also be formed of a biodegradable polymer, such as aliphatic polyesters, polycaprolactone, polybutylene succinate, poly(butylenes succinate-adipate), polylactic acid, terpolymer of terephthalic acid, adipic acid and 1,4-butanediol (such as ECOFLEX® from BASF Corp.), EASTAR® biodegradable aliphatic polyester available from Eastman Chemical Co., and biodegradable resin such as BIONOLLE® resin from Showa Highpolymer (Japan) and polylactic acid resins from Cargill Dow Polymers.

In addition U.S. Pat. 3,686,025 describes various spun bond sheets or webs of cellulosic and/or textile fibers that may comprise the fluid-impervious material.

The fluid-impervious material may be treated with antimicrobial agents and deodorants or perfumes. Examples of possible antimicrobial agents include methylparaben, ethylparaben, butylparaben, diazolidinyl urea, imidazolidenyl urea, miconazole nitrate, dichlorobenzyl alcohol, DMDM hydantoin, iodopropynyl butylcarbamate crystals, n-halamine, and secondary quarternary amines. The antimicrobials may be used alone or in compatible mixtures.

The wrapper 10 further includes a piece or length of deformable wire 16 attached to one side edge, such as by pressure-sensitive or hot melt adhesive. In an alternative embodiment, the wire 16 can be fully integrated into the wrapper 10, such as by encasing such wire 16 in thermoplastic before attaching to the wrapper 10, or placing such wire 16 between layers of the material making up the wrapper 10. In FIGS. 2 and 3, the wire 16 extends the entire width of the wrapper, with each wire tip projecting beyond the side edges of the wrapper 10. In another embodiment, the extended tips or ends of the wire 16 are encased in thermoplastic, adhesive, or other protective material, such as has been described in the art.

The wire 16 may be formed of any bendable material that will hold a fold or crimp, such as a metal, a metal coated with a thermoplastic or polymeric material, and a metal sandwiched between thermoplastic or paper sheets.

Fold or compression lines 18, 20 in the wrapper 10 are shown in FIGS. 2 and 3. As illustrated, the fold lines 18, 20 are spaced apart along the length of the wrapper 10 so that there is enough space between the lines 18 and 20 for a used feminine article, such as a tampon 22 (FIG. 3) or a sanitary napkin 24 (FIG. 3A) to be placed lengthwise atop the wrapper 10 and between the fold lines 18, 20. In one preferred embodiment, the fold lines 18 and 20 are spaced equidistant along the length of the wrapper 10, such as in a “C” fold configuration (see also FIG. 6).

The fold lines 18, 20 may be formed by physical folding or compression of the wrapper 10, or may also be formed by a variety of perforation means which have been described in the art. The means of forming the fold lines is not critical to the invention.

FIG. 3A depicts a disposal wrapper 10 with a used sanitary napkin 24 placed thereupon prior to discarding with the wrapper sheet described by the present invention. FIG. 3 depicts a disposal wrapper with a used tampon 22 placed thereupon prior to discarding. After placing a used feminine article 22 or 24 on the front face 14 (or rear face 12) of the wrapper 10, the wrapper 10 is folded first at the outermost fold line from the wire 16, so that the wrapper 10 nearly covers the used article.

Various methods of enclosing a used article 22 or 24 in a wrapper 10 are possible. As one example, the wrapper 10 may be folded at fold line 18 towards the partially-covered used article 22 so that the used article is completely covered by the wrapper 10. Alternatively, the wrapper 10 containing the used article 22 may then be turned upon itself using the fold line 18, whereby the wrapper 10 is again folded at fold line 18 in the direction of the wire 16 so that the used feminine article 22 is fully enclosed by the wrapper 10. That is, the wrapper 10 may be folded at fold line 18 in either direction. The side or bottom edge of the wrapper 10 containing the wire 16 will then overlay the rest of the wrapper which encloses the used article.

In an alternative embodiment, the wrapper 10 may be rolled in a continuous motion from the end furthest from the wire 16 over the used article 22 or 24 and towards the wire 16 until the used article 22 is completely enclosed by the wrapper 10, and the edge of the wrapper 10 in which the wire 16 is contained (or to which the wire 16 is attached) overlays the rest of the wrapper 10 which encircles the used article 22. The wire 16 then may be twisted at each end, between the portion of the wire which extends beyond the used feminine article 22 inside the wrapper 10 and the end of the wire.

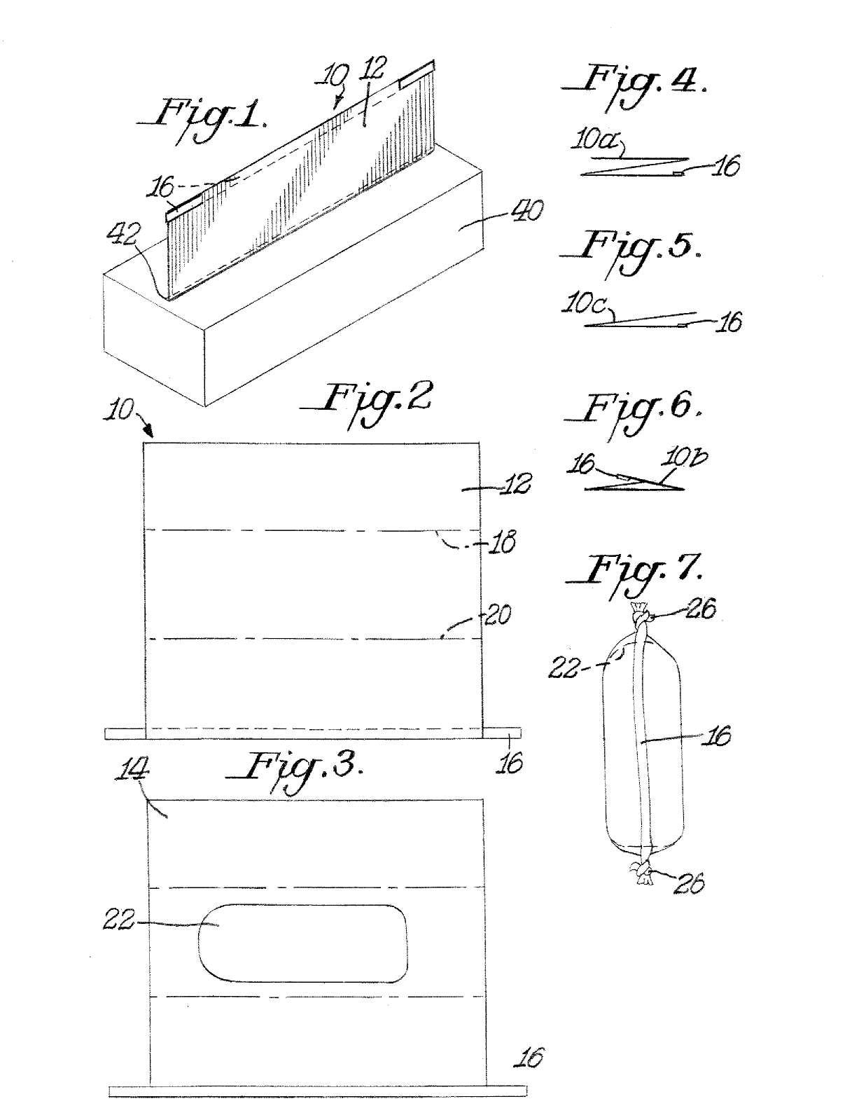

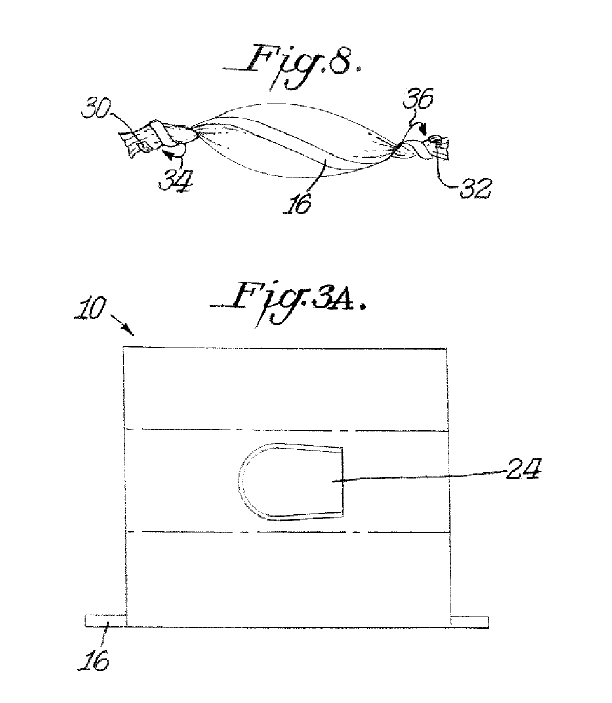

Used articles 22 or 24 encased within wrappers 10 are shown in FIGS. 7 and 8. FIG. 7 shows one embodiment in which each end of the wire 16 is twisted around itself and a portion of the wrapper material to form a loose knot 28. FIG. 8 shows a preferred embodiment in which each end 30, 32 of the wire 16 is twisted in opposing directions in the direction of arrows 34, 36. In this manner, the wrapper 10 can be pulled taught and further seal the used feminine article 22 or 24 within the wrapper 10 by means of further twisting one or both sides of the wire 34, 36.

FIG. 1 illustrates a dispenser carton 40 for multiple wrappers 10 of the present invention. The wrappers 10 nest within the carton 40. Individual wrappers 10 may be pulled upward and out of a top dispensing aperture 42 of the dispensing carton 40. Much like a facial tissue-box, the dispensing carton 40 contains several wrapper sheets which are stacked therein. In one embodiment as shown in FIG. 1, the wrappers 10 are exposed in a wire-end up orientation and protrude from the dispensing aperture 42.

While FIG. 1 shows a carton 40, alternate dispensers (not shown) may be used, such as a bag or plastic wrapper in which multiple wrappers 10 may be stored.

FIGS. 4, 5 and 6 illustrate alternate means for orienting folded wrappers 10a, 10b, 10c of the present invention for stacking in a dispenser carton 40. FIG. 4 depicts what is commonly referred to as a “Z-fold” 10a, whereby the wrapper 10a is folded twice, preferably at each fold line 18 and 20, with each fold in an opposite direction to form a “Z” shape. The wire 16 is either oriented at the top or bottom edge of the “Z” depending on the viewer's orientation. FIG. 5 depicts what is commonly referred to as a “V-fold” 10c, whereby the wrapper 10 is folded one time, frequently at or near the midpoint along its length, so that the wrapper 10 lays upon itself with the wire 16 oriented at either the top or bottom edge of the “V” depending on the viewer's orientation.

Whether the Z-fold 10a or V-fold 10c is utilized, the folded wrappers 10a, 10c can be stacked within the dispenser carton 40 so that they may be removed from the top aperture 42 of the dispenser carton 40. Wrappers 10 in either the Z-fold 10a or V-fold 10c configuration may be stacked in an interfolding manner so that adjacent wrappers 10 are in a nesting position, or stacked relation to one another. By nesting the wrappers 10, when one wrapper 10 is removed from the top aperture 42 of the dispenser carton 40, another wrapper is pulled upward so that it is partially exposed through the aperture or slot 42, and ready for removal. As shown in FIG. 1, the edge of the wrapper 10 with the wire 16 extends upwards through the aperture 42.

Alternatively, wrappers 10 in either the Z-fold 10a or V-fold 10c configuration may be stacked in a non-nesting manner within the dispensing carton 40. To dispense a wrapper 10a or 10c from the dispenser carton, the user reaches into the top aperture 42 and pulls out a wrapper 10 by the center of the wrapper 10, or alternatively the edge with the wire 16.

FIG. 6 depicts what is commonly referred to as a “C-fold” 10b, whereby the wrapper 10b is folded twice, preferably at each fold line 18 and 20, with each fold in the same direction to form a “C” shape. The wrapper 10b can be folded so that the wire 16 is oriented at the top of the folded wrapper 10b.

Wrappers 10b folded in a C-fold may be stacked within the dispenser carton 40. The C-fold 10b results in the edge containing the deformable wire 16 to be nearer the center of a stack of wrappers 10b, which may correspond to the location of the top aperture 42 of the disposal carton 40. To dispense a wrapper 10b from the dispenser carton 40, the user reaches into the top aperture 42 and pulls out a wrapper 10b by the edge containing the wire 16. In this way, the wire 16 may act as an aid for locating and gripping the edge of a wrapper 10b between a user's fingers to facilitate removal of the wrapper 10b from the disposal carton 40.

The invention has been illustrated by detailed description and examples of the preferred embodiments Various changes in form and detail will be within the skill of persons skilled in the art. Therefore, the invention must be measured by the claims and not by the description of the examples or the preferred embodiments.

I claim:

1. A disposal wrapper for used feminine articles, comprising:
a fluid-impervious sheet having a front face, a bottom face, a length, a width and at least one edge, and
a deformable wire at the at least one edge.

2. The disposal wrapper of claim 1 wherein the fluid-impervious sheet is formed from a material selected from the group consisting of: thermoplastic films, thermoplastic sheets, nonwovens coated with thermoplastic material, and laminates of thermoplastic with a nonwoven or nonwoven.

3. The disposal wrapper of claim 1 wherein the wire is formed from a material selected from the group consisting of: metal, metal coated with a polymeric material, and metal sandwiched between thermoplastic or paper sheets.

4. The disposal wrapper of claim 1 wherein the wire has a length that is greater than the width of the sheet.

5. The disposal wrapper of claim 1 wherein the wire has a length that is greater than the length of the sheet.

6. The disposal wrapper of claim 1 wherein the sheet defines one or more fold lines.

7. The disposal wrapper of claim 1 wherein the deformable wire is attached to the sheet.

8. The disposal wrapper of claim 1 wherein the sheet is formed as a laminate from to or more layers and the deformable wire is held between at least two of such layers.

9. The disposal wrapper of claim 1 wherein the sheet includes fold lines so that the sheet may be folded in at least one fold configuration selected from the group consisting of “C”-fold, “V”-fold and “Z”-fold.

10. A method of disposing a used feminine article, comprising
providing a wrapper having a fluid-impervious sheet having a front face, a bottom face, a length, a width and at least one edge, and a deformable wire at the at least one edge;
placing the used feminine article on one face of the sheet;
encircling the used feminine article with the sheet; and
securing the sheet over the used feminine article by twisting the deformable wire.

11. The method of claim 10, wherein the step of encircling the used feminine article with the sheet comprises rolling up the sheet around the used feminine article.

12. The method of claim 10, wherein the step of securing the sheet over the used feminine article comprises twisting each end of the deformable wire in an opposite direction.

13. The method of claim 10, wherein the step of securing the sheet over the used feminine article comprises twisting each end of the deformable wire to form a knot therein.

14. A dispenser carton for disposal wrappers for used feminine products, comprising
one or more walls defining an volume for holding multiple disposal wrappers, wherein at least one wall is a top wall defining an aperture to permit disposal wrappers to be pulled out of the volume; and
a plurality of disposal wrappers held in stacked relation within said volume, wherein each of said wrappers comprises a fluid-impervious sheet having a front face, a bottom face, a length, a width and at least one edge, and a deformable wire at the at least one edge.

15. The dispenser carton of claim 14, wherein the disposal wrappers are folded in a fold configuration selected from the group consisting of “V”-fold, “C”-fold and “Z”-fold.
